(19) United States
(12) Patent Application Publication
(10) Pub. No.: US 2011/0184270 A1
(43) Pub. Date: Jul. 28, 2011

(54) PHYSIOLOGICAL MONITORING GARMENT

(76) Inventors: Brian Russell, Crownsville, MD (US); Stephen Kent, Auckland (NZ)

(21) Appl. No.: 13/014,582

(22) Filed: Jan. 26, 2011

Related U.S. Application Data
(60) Provisional application No. 61/298,352, filed on Jan. 26, 2010.

(51) Int. Cl. A61B 5/04

(52) U.S. Cl. 600/388

(57) ABSTRACT

A garment for providing physiological monitoring of a wearer is provided. In one embodiment, the garment comprises a sensor configured to detect a physiological signal of a wearer; a stretchable member connected to a first connection point; an adjusting member connected to the stretchable member; an adjusting member configured to adjust a distance between the stretchable member and a second connection point; and wherein the stretchable member is configured to stretch when the distance is less than a predetermined distance to urge the sensor into contact with the wearer's skin.

### CROSS REFERENCE TO RELATED APPLICATIONS

This application claims priority to U.S. Provisional Application No. 61/298,352, filed Jan. 26, 2010, which is hereby incorporated by reference in its entirety for all purposes.

### FIELD OF INVENTION

The invention relates to a shirt or other garment with an integral or attached sensor or sensors for physiological monitoring that can be tightened to bring the sensors in contact with the wearer's skin.

### BACKGROUND

Monitoring of physiological signals of a person may be performed using a physiological sensor. Such sensors may be incorporated into a garment worn by the person such as a base layer shirt for example. However, movement of the wearer of the garment may cause the sensor to transiently move away from the wearer skin and prevent the reliable collection of physiological signals. However, it is not practical to custom make each garment to be sized to the person's body. Consequently, it may be desirable to incorporate a sensor into a garment that allows the wearer to adjust an adjustment mechanism of the garment to ensure reliable contact of the sensor(s) to the wearer's skin.

These and other advantages may be incorporated into embodiments of the present invention.

### BRIEF DESCRIPTION OF THE FIGURES

The invention is further described in the detailed description that follows, by reference to the noted drawings by way of non-limiting illustrative embodiments of the invention, in which like reference numerals represent similar parts throughout the drawings. As should be understood, however, the invention is not limited to the precise arrangements and instrumentalities shown. In the drawings:

FIG. 1 is a view of the front exterior of a shirt of a first embodiment of the invention which is a T-shirt,

FIG. 2 is a view of the internal side of the back of the shirt of FIG. 1,

FIG. 3 is an enlarged view of a portion of the internal side of the back of the shirt,

FIG. 4 is a view of the upper part of the front of a shirt of another embodiment of the invention, and

FIG. 5 is a view of the front exterior of a shirt of a yet another embodiment of the invention which is a long sleeved and collared shirt, and FIG. 5a shows in detail part of the shirt of FIG. 5.

### DETAILED DESCRIPTION OF EXAMPLE EMBODIMENTS

In the following description, for purposes of explanation and not limitation, specific details are set forth, such as particular materials, connectors, sensors, locations, garments, communication systems, computers, terminals, devices, techniques, interfaces, etc. in order to provide a thorough understanding of the present invention.

However, it will be apparent to one skilled in the art that the present invention may be practiced in other embodiments that depart from these specific details. Detailed descriptions of well-known materials, connectors, sensors, locations, garments, communication systems, computers, terminals, devices, techniques, interfaces, etc, are omitted so as not to obscure the description of the present invention.

In broad terms the invention comprises a garment for physiological monitoring, comprising one or more electrodes or sensors for detecting a wearer's physiological signal integral in the garment, or at least one connector integral in the garment for connecting to the garment an electrode or sensor for detecting a wearer physiological signal, an elastic section of the garment or associated with the garment, in the region of the electrode sensor(s), and a cord or cords (as herein subsequently described) for tightening the garment about a wearer to stretch the elastic section to bring the electrode(s) or sensor(s) in contact with the wearer's body under (light) pressure to form an electrical contact with the wearer's skin.

The garment may be a shirt, a separate sleeve (only—as worn by cyclists for example), or pants, for example.

Example embodiments of the present invention provide a garment that includes an adjustment mechanism which the wearer can adjust to stretch an elastic section to bring the sensor(s) into reliable contact with the user's skin.

Garments of example embodiments of the invention may comprise integrated physiological sensor(s) worn loosely but that can tensioned quickly and easily, to bring the electrode(s) into contact with the skin for detecting physiological signals. In at least embodiment, the garment may integrate the physiological sensor(s) such that they are not obvious from the exterior of the garment and so that the garment appears as a conventional sports or recreational garment, or work or uniform garment as worn by a first responder or for military use for example. When worn loose the garment does not interfere with the wearer's normal activity or comfort. In at least embodiment of a shirt of the invention the elastic section does not encircle the shirt and does include any discrete band separate from the material from which the shirt itself is formed, so that when worn there is no elastic section or discrete band which encircles the wearer's torso. When the shirt is tightened a non-encircling elastic section and a non-encircling adjustment strap also with part of the material or fabric from which the shirt itself is formed, together encircle the wearer's torso and are under tension bringing the electrode into contact with the wearer's body. This structure may provide greater user comfort, and may enable garments of some example embodiments of the invention to be lighter weight and the physiological monitoring system components of the garment less apparent, because there is not a fully encircling typically relatively thick elastic strap or band in or associated with the garment.

In broad terms in another aspect the invention comprises a shirt for physiological monitoring, comprising: one or more physiological signal detecting electrodes or sensors in a chest part of the shirt, or one or more connectors in the shirt for connecting to the garment a physiological signal detecting electrode or sensor in a chest part of the shirt, an elastically stretchable section or sections of the shirt in a chest part of the shirt or in a back or side part or opposite side parts of the shirt at a height of the chest part of the garment, said elastically stretchable section not encircling the shirt, and an adjustment strap or straps around the chest part of the shirt or a back or side part or opposite side parts of the shirt at a height of the chest part of the shirt, said strap or straps also not encircling the shirt, for tightening the shirt about a wearer at chest height stretching the elastically stretchable section(s), to bring the electrode(s) or sensor(s) in contact with the wearer's chest under to form an electrical contact with the wearer's skin.

As used herein the term “shirt” includes a shirt with or without sleeves whether short or long, and with or without a collar, such as a sleeveless vest, a T shirt, a collared shirt, or a collared and long sleeved shirt for example, of any material such as a lightweight material for sports use or a heavier material for first responder or military use, and refers to any base layer garment.

As used herein the term “tight” refers to a fit on the body providing good or satisfactory sensor contact (to permit reliable sensing) to the skin without adversely restricting movement or normal bodily function.

As used herein the term “loose” refers to a fit on the body that does not exert enough tension for good or satisfactory sensor contact (to permit reliable sensing), but a garment when loose may still be a form fitting or compression garment that can be further tensioned when required to a tight fit as defined above.

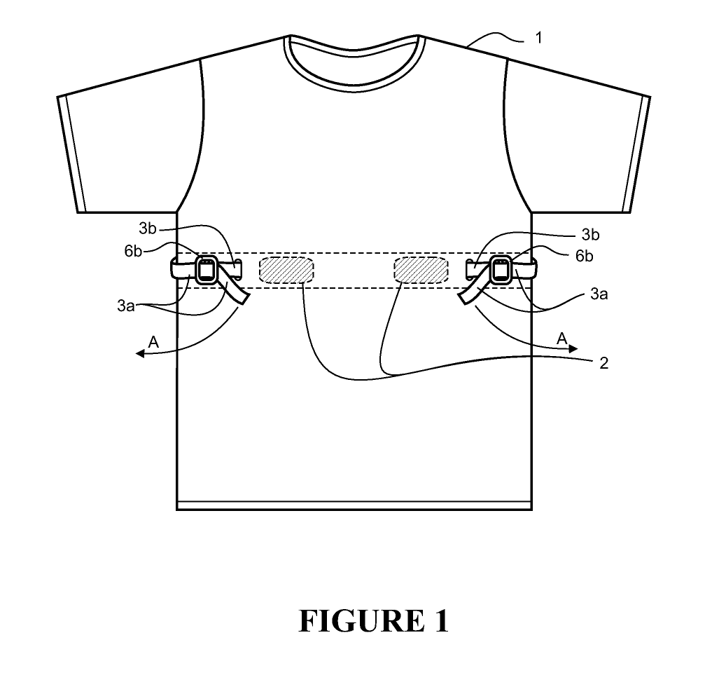

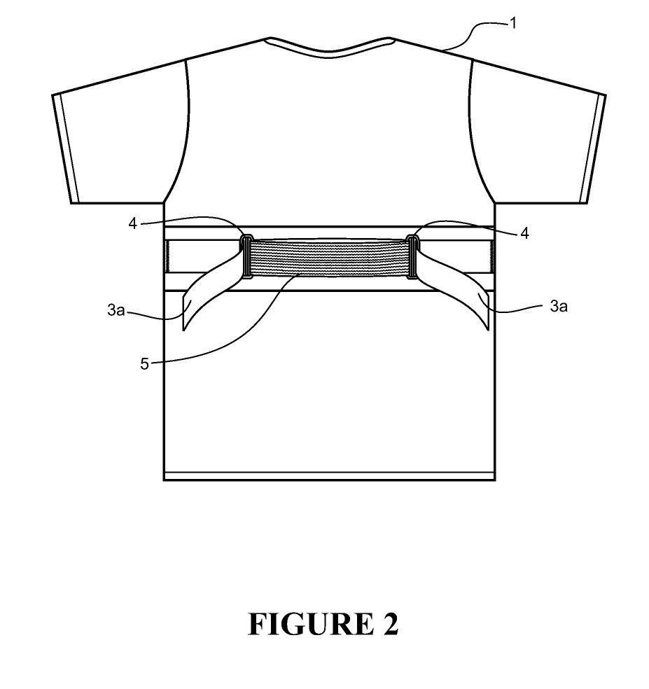

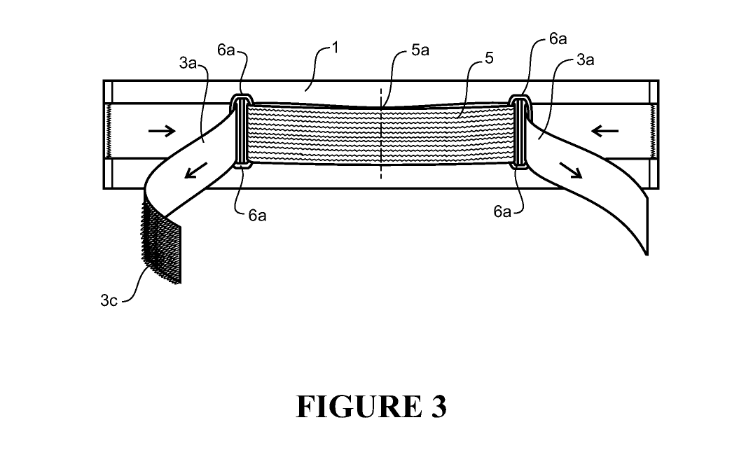

Referring to FIGS. 1 to 3, a garment 1 such as a T-shirt has one or more physiological monitoring electrodes or sensors 2 at chest height (by height is meant the dimension from the bottom of the body of the shirt to the shoulders of the shirt) such as heart rate, temperature, skin conductance or resistance or reactance, breathing rate, activity, posture, SPO2, or impact sensors for example, integral in the front of the shirt. The sensors may be discrete sensors fixed to the interior of the front of the shirt or may comprise area(s) of the fabric from which the shirt is formed that comprise conductive fibres, for example. A sensor may incorporate a resiliently compressible layer such as a flexible foam, air, liquid or gel layer between the garment fabric and the contact face of the electrode, which may also have some shear flexibility for shear movement absorption across the electrode, to assist in isolating the contact face of the electrode (against the wearer's skin in use) from movement of the garment fabric or body of the garment. Alternatively the garment on the interior of the front may integrally incorporate one or more separate connectors to which one or more electrodes or sensors may be attached, and removed for laundering the garment for example. Alternatively again or additionally, sensors may be provided in one or both sides and/or the back of the garment (and/or in one or both sleeves such as at the forearm part of a long sleeve in long sleeved shirt embodiments). In the back of the garment (see FIGS. 2 and 3) at chest height there is an integral elastically stretchable section 5 of the shirt such as strip or panel stitched to or otherwise attached to or incorporated in the shirt. The elastically stretchable section 5 does not encircle the shirt so that when worn it will not encircle the wearer's torso. Alternatively or additionally an elastically stretchable section 5 may be provided in the front of the shirt and/or in one or both sides of the shirt (but a continuous elastically stretchable section does not encircle the shirt of this embodiment).

On each side of the shirt a strap 3b is connected to the front of the shirt at chest height and approximately the height of the sensors 2, and on each side an adjustment strap 3a is connected to the side or back of the shirt, and on each side the two straps 3a and 3b are connected by strap connector 6b such as a buckle, clip, clamp, or similar, which in this embodiment comprises a lightweight plastic friction buckle. The straps 3a may pass from the inside to the outside of the garment through holes. The arrangement is such that when the shirt is loose on a wearer, the adjustment straps 3a can be pulled as indicated by arrows A sufficiently to stretch the elastic section(s) 5 and reduce the circumference of the shirt until it is tight about the chest and the sensors 2 are pressed against the body to detect physiological signal(s) (while the elastic section 5 allows the wearers chest to expand during normal respiration).

The ends of the adjustment straps 3a may be attachable after tightening to the outside of the garment by hook and loop fastening (velcro) between the strap end(s) and the garment alternative to the buckles 6b, buttons, or another fixing or locking mechanism for attaching two materials through a mechanical bond that can be separated via mechanical force in a direction different to that which keeps it together. When worn loosely the adjustment straps, buckles and sensors are not well seen as they can be hidden inside small pockets in the shirt and/or the strap ends may be at least partly under the arms preserving the look of the shirt.

Pulling the adjustment straps 3a to tighten the garment is quick and easy for a user. Similarly loosening the garment is simple, through manual release of the straps 3a or through the use of a push button release for example. Also, a user can easily don and doff the loose garment.

Electronics are used to read signals from the sensors. The electronics are small and may be connected to the sensor via conductors integrally incorporated into the garment, and/or connectors. An electronics module may be separate from the garment so the garment can be laundered and the electronics can be charged. To carry and conceal the electronics module a pocket may be provided on the exterior or interior side of the garment. The pocket may also protect the electronics module from being knocked off the shirt, and may hide the electronics from view so the shirt look is not compromised. Alternatively the electronics module(s) if carried by the garment can be placed anywhere on the garment such as in a pocket on the sleeve.

In more detail, and referring specifically to FIG. 3, the ends of straps 3a may be stitched into a side seam of the garment on either side, or the rear of the garment, and the straps 3a then extend around the interior side of the rear of the garment, towards the elastic section 5 at the centre of the rear of the garment. Either end of the elastic section 5 is coupled to one loop of an double loop component 4 for example moulded from a lightweight plastic, or may be otherwise fixed to the garment, and the strap 3a on either side passes loosely (non-friction lock) through the other loop of the part 4. The strap 3a then reverses direction and extends back around the rear and sides of the garment to the front of the garment (see FIG. 1) and through friction lock buckle 6b. In FIG. 3 a section 3c of one side of a hook and loop fastener (velcro) is shown on the end of one of the straps 3a, by which the loose end of the strap 3a may be attached to the front of the garment.

In the embodiment described and shown, when tightened the straps are held tightened by the friction buckles, clips, or clamps, lock and loop fastening 6b at the front of the garment on either side but alternatively these could be excluded and friction lock buckles or similar provided only at the rear of the garment at 6a on either side.

The straps 3 may alternatively comprise tapes, cords or other means of providing tension; no cross-section dimension or shape is limited or inferred by the use of the word strap.

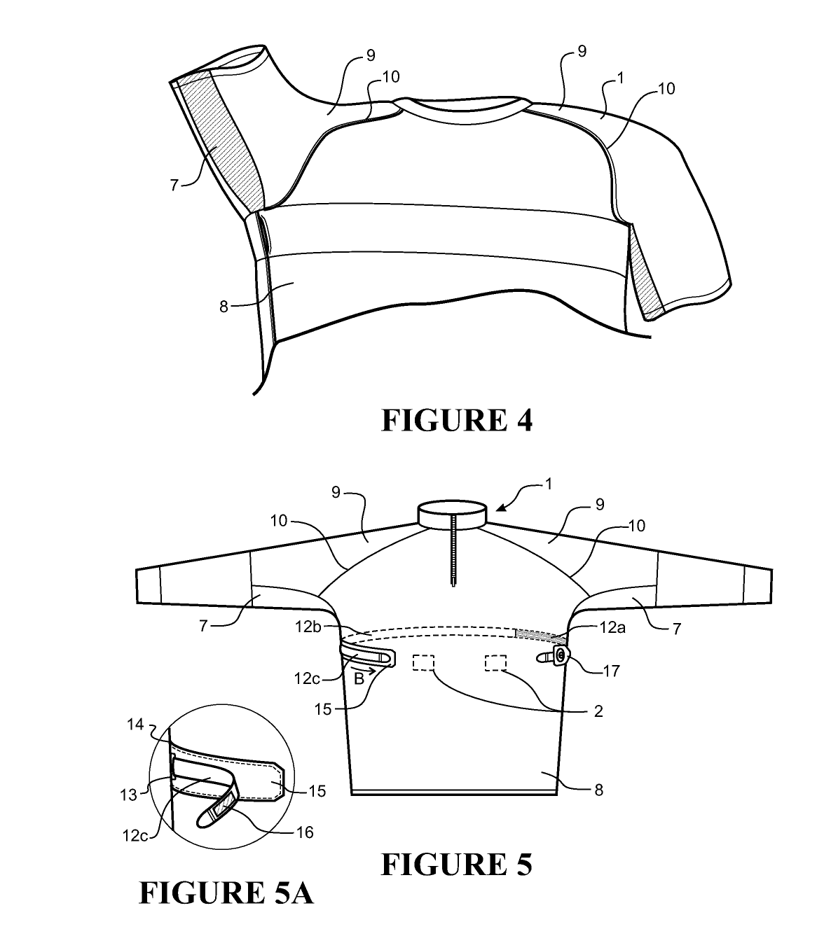

Motion artifacts and/or electrical noise can be introduced by movement between the sensor(s) and the skin, and may be worse for sensors carried by a garment rather than an encircling strap. To minimise garment movement leading to sensor movement and thus signal noise some embodiments of shirts of the invention may include underarm gussets 7 as in FIG. 4 to allow maximum movement of the arms without or with reduced movement of the body particularly front panel 8 of the shirt, which may cause movement of the sensor(s). When the arm moves upwards slack in the gusset 7 isolates (reduces movement of) sensors in the chest of the garment.

Some embodiments of shirts of the invention may also comprise saddle shoulders as also shown in FIG. 4. A saddle shoulder does not have a seam on the top of the shoulder of the shirt, which may tend to fall to the front or the back of the shoulder affecting the position or movement of the shirt as a whole and thus of the sensors on the body. A saddle shoulder comprises a panel 9 of fabric over the shoulder, stitched to the main front and back panels of the shirt by a front and back seam at a similar height, each from the neck to the underarm, a front seam 10 being shown in FIG. 4. Also using flat-stitching of the seams reduces the likelihood of the shirt catching on the body causing movement. Generally garment panels may be shaped and materials selected to maximise free movement of some parts of the garment while restricting movement of parts of the garment proximate to the electrode(s). For example chest panels may be of a stretchable material to aid fit and movement of the upper body while without movement of the sensor(s). Panels around the sensor area below the chest may be constructed of stronger elastic helping to retain sensors in place. Also areas of materials such as neoprene rubber or similar materials may be provided around sensor(s) to grip the skin to aid the sensors to hold their position on the body.

FIG. 5 is a view of the front exterior of a shirt of another embodiment of the present invention, and FIG. 5a provides a more detailed view of a part of the shirt of FIG. 5. The shirt is a long sleeved shirt with a collar as may be worn as a base layer garment by a first responder or military personnel. The shirt comprises physiological monitoring sensors 2 in the interior of the front of the shirt, as in the embodiment of FIGS. 1-3. In this embodiment a single strap 12 is fixed at one elastically stretchable end 12a to the interior of the shirt at the back as shown, such as by stitching, and in the embodiment shown at the back on one side, and comprises a longer free part 12b which passes around the interior of the back of the shirt at chest height, and passes out to the exterior of the shirt through a hole 13 for example like a buttonhole, which in the embodiment shown is in a side seam 14 as shown in detail in FIG. 6a, and passes to the front of the shirt on one side where the strap terminates in a free end 12c as shown. The elastically stretchable part 12a is shaded. The part 12b of the strap which is not fixed to the body of the shirt, and the free end 12c, are not elastic and are shown unshaded. A panel 15 of loop material of hook and loop fastening (velcro) is provided on the front exterior of the shirt at one side, and hook material 16 (velcro) is provided on the underside of the free end 12c of the strap exposed at the front exterior of the shirt on the same side, as shown in detail in FIG. 6b. To tighten the shirt for physiological monitoring, the wearer frees the distal end of the strap from the front of the shirt, and pulls the strap as indicated by arrow B, to tighten the chest part of the shirt around the wearer's body, and then reaffixes the end 12c of the strap to the front of the shirt via velcro to hold the shirt tight. To loosen the shirt the wearer does the opposite. Also in this embodiment the elastically stretchable part 12a and the strap parts 12b and 12c do not encircle the shirt. When the shirt is tightened the elastically stretchable part 12a, the strap parts 12b and 12c, and part of the body of the shirt itself (the material from which the shirt is formed) together encircle the wearer's torso and are under tension bringing the electrode into contact with the wearer's body.

As before an electronics module indicated at 17 may be attached to the garment or a pocket may be provided in the garment for containing the electronics module.

In another embodiment an electrode or electrodes such as a heart rate and/or temperature electrodes, and a single strap system as described above, are incorporated in the sleeve of a garment such as the upper arm part of the sleeve or the forearm part or wrist part of the sleeve or in a leg of a pants garment. Again by pulling the free end of the adjustment strap sufficiently and then reaffixing it to the sleeve or pants leg the wearer can tighten the garment sufficiently for sensor contact when the wearer desires to carry out physiological monitoring, and subsequently the wearer can release the strap to loosen the garment.

Thus, one embodiment may comprise a shirt for providing physiological monitoring of a wearer, that comprises a sensor configured to detect a physiological signal of the wearer; a first strap connected to a first connection point; a second strap connected to a second connection point; a stretchable member having a first end connected to the first strap and having a second end connected to the second strap; and the first strap having an adjustment mechanism configured to adjust a distance between the stretchable member and a second connection point to stretch the stretchable member to urge the sensor into contact with the wearer's skin. The sensor, stretchable member, first strap and second strap may located in a chest part of the shirt or a sleeve part. The shirt may comprise a sensor connector fixedly attached to the shirt and wherein the sensor is configured to be removably attached to the sensor connector to thereby permit removal and replacement of the sensor. The stretchable member may be located on an inside of the shirt and wherein the first strap extends from the first connection point inside the shirt through an aperture to an outside of the shirt. The adjustment mechanism may be configured to adjust a distance between the stretchable member and the first connection point; and wherein the stretchable member is configured to stretch when the distance is less than a predetermined distance to urge the sensor into contact with the wearer's skin.

In yet another embodiment, the invention may comprise a garment for providing physiological monitoring of a wearer, that comprises a sensor configured to detect a physiological signal of the wearer; a stretchable member connected to a first connection point; a first strap connected to a second connection point and connecting the second connection point to the stretchable member; and wherein the first strap having an adjustment mechanism configured to allow tightening of the first strap to stretch the stretchable member to bring the sensor in contact with the wearer's skin. The garment may include a sensor connector fixedly attached to the garment and wherein the sensor is configured to be removably attached to the sensor connector to thereby permit removal and replacement of the sensor. The garment may further include a second strap connecting the stretchable member to the first connection point. The stretchable member may be located on an inside of the garment and wherein the first strap extends from the second connection point inside the garment through an aperture to an outside of the garment. The adjustment mechanism may be configured to adjust a distance between the stretchable member and the second connection point; and wherein the stretchable member is configured to stretch when the distance is less than a predetermined distance to urge the sensor into contact with the wearer's skin.

In yet another embodiment, a garment for providing physiological monitoring of a wearer, may comprise a sensor configured to detect a physiological signal of a wearer; a stretchable member connected to a first connection point; an adjusting member connected to the stretchable member; an adjusting member configured to adjust a distance between the stretchable member and a second connection point; and wherein the stretchable member is configured to stretch when the distance is less than a predetermined distance to urge the sensor into contact with the wearer's skin. The adjusting member may comprise a strap extending from the stretchable member on an inside of the garment through an aperture to an outside of the garment. The adjusting member may comprise a first strap, the garment further comprising a second strap connecting the stretchable member to the first connection point; and wherein the first strap and the second strap are not stretchable. The sensor, adjusting member, stretchable member may be located in a back chest part of the garment.

In yet another embodiment, the invention may comprise a garment for physiological monitoring, comprising one or more electrodes or sensors for detecting a wearer physiological signal integral in the garment, or at least one connector integral in the garment for connecting to the garment an electrode or sensor for detecting a wearer physiological signal, an elastic section of the garment or associated with the garment, proximate to the electrodes or sensors, and a strap or straps directly or indirectly between the part or parts of the garment in which the sensor(s) are located and the elastic section of the garment for tightening the garment about a wearer while stretching the elastic section to bring the electrode(s) or sensor(s) in contact with the wearer's body under pressure to form an electrical contact with the wearer's skin. The sensors, elastic section, and strap or straps may be in a chest part of the garment, or in a chest part and a back or side part or opposite side parts of the garment at a height of the chest part of the garment.

In yet another embodiment, the invention may comprise a shirt for physiological monitoring, comprising: one or more physiological signal detecting electrode(s) or sensor(s) in a chest part of the shirt, or one or more connectors in the shirt for connecting to the garment a physiological signal detecting electrode or sensor in a chest part of the shirt, an elastically stretchable section or sections of the shirt in a chest part of the shirt or in a back or side part or opposite side parts of the shirt at a height of the chest part of the garment, the elastically stretchable section not encircling the shirt, and an adjustment strap or straps around the chest part of the shirt or a back or side part or opposite side parts of the shirt at a height of the chest part of the shirt, the strap or straps also not encircling the shirt, for tightening the shirt about a wearer at chest height stretching the elastically stretchable section(s), to bring the electrode(s) or sensor(s) in contact with the wearer's chest under to form an electrical contact with the wearer's skin. The elastically stretchable section may comprise a transversely extending strip integral in the shirt. The elastically stretchable section may integral in a back part of the shirt. The elastically stretchable section may be integral in a front part of the shirt. The elastically stretchable section may be in a front part of the shirt. The elastically stretchable section may be in one or both sides of the shirt. The adjustment strap may extend from a rear part or rear side part of the shirt to a fastener for an opposite end of the strap at a front part or front side part of the shirt on a left or right hand side of the shirt only. The adjustment strap may extend from a rear part or rear side part of the shirt to a fastener for an opposite end of the strap at a front part or front side part of the shirt on both a left and a right hand side of the shirt. Each side of the shirt may have an adjustment strap that extends from an end of the strap fixed to the shirt, to a rear part or rear side part of the shirt and through a loop fixed to the shirt at rear part or rear side part of the shirt, from the loop to a fastener for an opposite end of the cord at a front part or front side part of the shirt. The end of each the adjustment strap may be fixed to the shirt such as stitched into a side seam of the shirt. The shirt may comprise saddle shoulders, sleeves, underarm gussets. The shirt may comprise a fastener for releasably fixing a free end of the adjustment strap to the shirt when adjusted to tighten the chest part of the shirt about a wearer to stretch the elastically stretchable section and bring the electrode or sensor in contact with the wearer's body under pressure to form an electrical contact with the wearer's skin.

It is to be understood that the foregoing illustrative embodiments have been provided merely for the purpose of explanation and are in no way to be construed as limiting of the invention. Words used herein are words of description and illustration, rather than words of limitation. In addition, the advantages and objectives described herein may not be realized by each and every embodiment practicing the present invention. Further, although the invention has been described herein with reference to particular structure, materials and/or embodiments, the invention is not intended to be limited to the particulars disclosed herein. Rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention.

What is claimed is:

1. A shirt for providing physiological monitoring of a wearer, comprising:
a sensor configured to detect a physiological signal of the wearer;
a first strap connected to a first connection point;
a second strap connected to a second connection point;
a stretchable member having a first end connected to said first strap and having a second end connected to said second strap;
said first strap having an adjustment mechanism configured to adjust a distance between said stretchable member and a second connection point to stretch said stretchable member to urge said sensor into contact with the wearer's skin.

2. The shirt according to claim 1, wherein said sensor, said stretchable member, said first strap and said second strap are located in a chest part of the shirt.

3. The shirt according to claim 1, wherein said sensor, said stretchable member, said first strap and said second strap are located in a sleeve part of the shirt.

4. The shirt according to claim 1, further comprising a sensor connector fixedly attached to the shirt and wherein said sensor is configured to be removably attached to said sensor connector to thereby permit removal and replacement of said sensor.

5. The shirt according to claim 1, wherein said second strap includes an adjustment mechanism.

6. The shirt according to claim 1, wherein said stretchable member is located on an inside of the shirt and wherein said first strap extends from said first connection point inside the shirt through an aperture to an outside of the shirt.

7. The shirt according to claim 1, wherein said adjustment mechanism configured to adjust a distance between said stretchable member and said first connection point; and
wherein said stretchable member is configured to stretch when the distance is less than a predetermined distance to urge said sensor into contact with the wearer's skin.

8. A garment for providing physiological monitoring of a wearer, comprising:
a sensor configured to detect a physiological signal of the wearer;
a stretchable member connected to a first connection point;
a first strap connected to a second connection point and connecting said second connection point to said stretchable member; and
said first strap having an adjustment mechanism configured to allow tightening of said first strap to stretch said stretchable member to bring said sensor in contact with the wearer's skin.

9. The garment according to claim 8, wherein said sensor, said stretchable member, and said first strap are located in a chest part of the garment.

10. The garment according to claim 8, wherein said sensor, said stretchable member, and said first strap are located in a sleeve part of the garment.

11. The garment according to claim 8, further comprising a sensor connector fixedly attached to the garment and wherein said sensor is configured to be removably attached to said sensor connector to thereby permit removal and replacement of said sensor.

12. The garment according to claim 8, further comprising a second strap connecting said stretchable member to said first connection point.

13. The garment according to claim 8, wherein said stretchable member is located on an inside of the garment and wherein said first strap extends from said second connection point inside the garment through an aperture to an outside of the garment.

14. The garment according to claim 8, wherein said adjustment mechanism is configured to adjust a distance between said stretchable member and said second connection point; and
wherein said stretchable member is configured to stretch when the distance is less than a predetermined distance to urge said sensor into contact with the wearer's skin.

15. A garment for providing physiological monitoring of a wearer, comprising:
a sensor configured to detect a physiological signal of a wearer;
a stretchable member connected to a first connection point;
an adjusting member connected to said stretchable member;
an adjusting member configured to adjust a distance between said stretchable member and a second connection point; and
wherein said stretchable member is configured to stretch when the distance is less than a predetermined distance to urge said sensor into contact with the wearer's skin.

16. The garment according to claim 15, wherein said adjusting member comprises a strap extending from said stretchable member on an inside of the garment through an aperture to an outside of the garment.

17. The garment according to claim 15, wherein said adjusting member comprises a first strap, the garment further comprising a second strap connecting said stretchable member to said first connection point; and
wherein said first strap and said second strap are not stretchable.

18. The garment according to claim 15, further comprising a pocket configured to hold a processing device configured to be connected to said sensor.

19. The garment according to claim 15, further comprising a sensor connector fixedly attached to the garment and wherein said sensor is configured to be removably attached to said sensor connector to thereby permit removal and replacement of said sensor.

20. The garment according to claim 15, wherein said sensor, said adjusting member, said stretchable member are located in a back chest part of the garment.
